(19) United States
(12) Patent Application Publication
(10) Pub. No.: US 2005/0277187 A1
(43) Pub. Date: Dec. 15, 2005

(54) Creation of shear in a reactor

(75) Inventors: Timothy J. Johnson, Andover, MA (US); Bernardo D. Aumond, Marblehead, MA (US); Brian O. Benoit, Woburn, MA (US); George J. Vella, Medway, MA (US); Seth T. Rodgers, Somerville, MA (US); Andrey J. Zarur, Winchester, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: BioProcessors Corp., Woburn, MA (US)

(21) Appl. No.: 11/147,416

(22) Filed: Jun. 7, 2005

Related U.S. Application Data
(60) Provisional application No. 60/636,420, filed on Dec. 14, 2004.
(60) Provisional application No. 60/577,986, filed on Jun. 7, 2004.
(60) Provisional application No. 60/577,977, filed on Jun. 7, 2004.

(51) Int. Cl. B01J 19/00; C12M 1/02

(52) U.S. Cl. 435/293.1; 422/102; 422/130; 435/297.5

(57) ABSTRACT

An apparatus for performing a biological or biochemical reaction that, in certain embodiments, has the ability to apply shear stress to a component of a liquid sample and includes a biological or biochemical reactor comprising a container having a volume of less than about 2 mL and containing a liquid sample, and a shear-generating element, the shear-generating element being contained within the apparatus and constructed and arranged so that the entire shear-generating element moves along a selected path of motion intersecting a first location within the apparatus and a second location within the apparatus, with or without rotational movement is described. A method of applying shear stress to a component of a liquid sample that includes moving a liquid or gaseous shear-generating element within an apparatus along a selected path of motion to create a reproducible and controllable level of shear stress at a selected location within the liquid sample is also disclosed.

### RELATED APPLICATIONS

This application claims priority under 35 U.S.C. § 119(e) to U.S. Provisional Application Ser. No. 60/577,977, entitled “Gas Control in a Reactor,” filed on Jun. 7, 2004, U.S. Provisional Application Ser. No. 60/577,986, entitled “Reactor Mixing,” filed on Jun. 7, 2004, and U.S. Provisional Application Ser. No. 60/636,420, entitled “Creation of Shear in a Reactor,” filed on Dec. 14, 2004, each of which is herein incorporated by reference in its entirety.

### FIELD OF INVENTION

The present specification discloses the creation of shear forces in reaction systems, and in certain embodiments the creation of shear forces to affect the behavior of biological cells.

### DESCRIPTION OF RELATED ART

Cells are cultured for a variety of reasons. Increasingly, cells are cultured for proteins or other valuable materials they produce. Typically, cells require specific conditions be maintained for viability and/or optimal growth and/or productivity, maintenance of such conditions as with a controlled environment can be necessary or advantageous for many cell cultures. The presence of nutrients, metabolic gases such as oxygen and/or carbon dioxide, proper levels of humidity, as well as control of other factors such as temperature, may affect cell growth and/or cell behavior. Cells require time to grow, during which favorable conditions should be maintained. In some cases, such as with particular bacterial cells, a successful cell culture may be performed in as little as 24 hours. In other cases, such as with particular mammalian cells, a successful culture may require about 30 days or more.

Typically, cell cultures are performed in media suitable for cell growth and containing necessary nutrients. The cells are generally cultured in a location, such as an incubator, where the environmental conditions can be controlled. Incubators traditionally may range in size from small incubators (e.g., about 1 cubic foot or less) for a few cultures and/or small culture volumes up to an entire room or rooms in which the desired environmental conditions can be carefully maintained.

More generally, a wide variety of reaction systems are known for the production of products of chemical reactions, biochemical reactions, and/or biological systems. Chemical plants involving catalysis, biochemical fermenters, pharmaceutical production plants, and a host of other systems are well-known. Biochemical processing may involve the use of a live microorganism (e.g., cells) to produce a substance of interest.

As described in U.S. patent application Ser. No. 09/707,852, filed on Nov. 7, 2000, entitled “Microreactor,” incorporated herein by reference, cells have also been cultured on a very small scale (i.e., on the order of a few milliliters of culture volume or less), so that, among other things, many cultures can be performed in parallel.

While important and valuable advances have been made in the field of cell culture and other fields, improvements would be valuable.

### SUMMARY

Each of the following commonly-owned applications directed to related subject matter and/or disclosing methods and/or devices and/or materials useful or potentially useful for the practice of the present invention is incorporated herein by reference: U.S. patent application Ser. No. 10/457,017, filed Jun. 5, 2003, entitled “System and Method for Process Automation,” by Rodgers, et al.; U.S. patent application Ser. No. 10/457,049, filed Jun. 5, 2003, entitled “Materials and Reactor Systems having Humidity and Gas Control,” by Rodgers, et al,. published as 2004/0058437 on Mar. 25, 2004; U.S. patent application Ser. No. 10/457,015, filed Jun. 5, 2003, entitled “Reactor Systems Having a Light-Interacting Component,” by Miller, et al., published as 2004/0058407 on Mar. 25, 2004; U.S. patent application Ser. No. 10/456,929, filed Jun. 5, 2003, entitled “Apparatus and Method for Manipulating Substrates,” by Zarur, et al.; U.S. patent application Ser. No. 10/664,046, filed Sep. 16, 2003, entitled “Determination and/or Control of Reactor Environmental Conditions,” by Miller, et al., published as 2004/0132166 on Jul. 8, 2004; U.S. patent application Ser. No. 10/664,068, filed Sep. 16, 2003, entitled “Systems and Methods for Control of pH and Other Reactor Environmental Conditions,” by Miller, et al., published as 2005/0026134 on Feb. 3, 2005; U.S. patent application Ser. No. 10/664,067 filed on Sep. 16, 2003, entitled “Microreactor Architecture and Methods,” by Rodgers, et al.; and U.S. Patent Application Ser. No. 60/577,985 filed on Jun. 7, 2004, entitled “Control of Reactor Environmental Conditions,” by Rodgers, et al.

The present specification discloses chemical, biological, and/or biochemical reactor chips and/or reaction apparatuses and associated systems such as microreactor systems. The subject matter of this invention involves, in some cases, interrelated products, alternative solutions to a particular problem, and/or a plurality of different uses of one or more systems and/or articles.

According to one embodiment of the invention, an apparatus for performing a biological or biochemical reaction having the ability to apply shear stress to a component of a liquid sample, comprises a biological or biochemical reactor comprising a container having a volume of less than about 2 mL, the container containing a liquid sample, the apparatus also includes a shear-generating element, which does not comprise a surface of a container or a conduit in contact with a liquid, the shear-generating element being contained within the apparatus and constructed and arranged so that the entire shear-generating element moves along a selected path of motion intersecting a first location within the apparatus and a second location within the apparatus, with or without rotational movement.

According to another embodiment of the invention, an apparatus for performing a biological or biochemical reaction having the ability to apply shear stress to a component of a liquid sample comprises a biological or biochemical reactor comprising a container containing a liquid sample. The apparatus also comprises a shear-generating element, which does not comprise a surface of a container or a conduit in contact with a liquid, the shear-generating element being contained within the container and constructed and arranged so that the entire shear-generating element moves along a selected path of motion intersecting a first location within the container and a second location within the container. In this embodiment, changes in the movement of the shear-generating element within the container that create changes in a level or pattern of shear stress in the liquid sample do not significantly affect gas exchange between the liquid sample and the exterior of the reactor.

According to yet another embodiment of the invention, an apparatus for performing a biological or biochemical reaction having the ability to apply shear stress to a component of a liquid sample comprises a biological or biochemical reactor comprising a container, the container containing a liquid sample. The apparatus also comprises a shear-generating element within the apparatus that is movable within the apparatus upon inversion of the apparatus, and a control system configured to control movement of the shear-generating element to facilitate creation of a reproducible and controllable level of shear stress at a selected location within the liquid sample.

According to another embodiment of the invention, a method of applying shear stress to a biological or biochemical component of a liquid sample contained within a container comprises moving and/or controlling movement of a shear-generating element within a container containing a liquid sample, the movement of the shear-generating element occurring upon inversion of the container, wherein the movement applies a reproducible and controllable level of shear stress to a biological or biochemical component at a selected location within the liquid sample.

In another embodiment of the invention, an apparatus for performing a biological or biochemical reaction having the ability to apply shear stress to a component of a liquid sample comprises a biological or biochemical reactor comprising a container configured to contain a liquid sample, a surface of the container comprising a membrane having an oxygen permeability of greater than or equal to 0.061 mol O₂/(day·m2·atm). The apparatus further comprises a shear-generating element contained within the container and constructed and arranged so that the entire shear-generating element moves along a selected path of motion intersecting a first location within the container and a second location within the container during operation when the container contains the liquid sample.

In yet another embodiment of the invention, a method of applying shear stress to a biological or biochemical component of a liquid sample comprises moving an entire shear-generating element, freely suspended within an apparatus, along a selected path of motion intersecting a first location within the apparatus and a second location within the apparatus to apply a reproducible and controllable level of shear stress to a biological or biochemical component at a selected location within a liquid sample, wherein the shear-generating element is either a gas or a liquid.

According to a further embodiment of the invention, an apparatus for performing a biological or biochemical reaction having the ability to apply shear stress to a component of a liquid sample comprises a biological or biochemical reactor comprising a container having a volume of less than about 2 mL and containing a liquid sample. The apparatus also comprises a shear-generating element, which does not comprise a surface of a container or a conduit in contact with a liquid, the shear-generating element being contained within the container and constructed and arranged for pivoting movement within the container, the pivoting movement creating a reproducible and controllable level of shear stress at a selected location within the liquid sample.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1 illustrates a layer of a chip including six reactors including reaction site containers that can be used in accordance with one embodiment of the invention;

FIGS. 2a-2c illustrate various orientations in which chips may be positioned on a rotating apparatus;

FIGS. 3a-3c show selected movement directions of shear-generating elements within containers;

FIG. 4 shows one illustrative embodiment of a rotating apparatus and control system that can be used in accordance with the invention;

FIG. 5a shows one illustrative embodiment of a shear-generating element that is slidingly attached to a container;

FIG. 5b shows one illustrative embodiment of a shear-generating element that is pivotally attached to a container;

FIG. 6 shows a perspective view of a container having a thickness that varies along the path of movement of a shear-generating element;

FIG. 7 shows a graph of the shear stress created by a shear-generating element comprising a gas bubble moving through the reaction site container shown in FIG. 6, as simulated via computational fluid dynamics modeling; and

FIG. 8 shows a top view of a strain rate contour plot of a planar cross-section taken along line VIII-VIII shown in FIG. 6, at ninety degrees of rotation.

### DETAILED DESCRIPTION

The present specification discloses chemical, biological, and/or biochemical reactor chips and/or reaction systems such as microreactor systems, as well as systems and methods for using such devices. In certain embodiments of the invention, a chip, a reactor, or a reaction system containing a liquid sample may be configured for reproducibly controlling and/or creating shear stress within a reaction site container (hereinafter also referred to as simply “container”), such as a cell culture chamber, for example in order to subject cells to particular shear stress. Shear stress can have a dramatic effect on the behavior of many types of biological cells by altering, for example, one or more of protein production, gene expression, cell morphology, or likelihood of cell death. The shear stress may be created with a shear-generating element such as, for example, a gas bubble, a solid bead (e.g. a glass or plastic bead) a magnetically-activated element (e.g. a magnetic bead), and/or a liquid bolus that is immiscible with the liquid sample.

Certain chemical and pharmaceutical bioreactors, including large scale bioreactors, expose biological cells to hydrodynamic shear stress via mixing impellers and/or gas sparging, and/or various other means of pumping and/or mixing. Because of the various effects of shear stress on biological cells in these bioreactors, the successful operation of a bioreactor may depend on the creation of an appropriate amount of hydrodynamic shear. Data obtained from microreactor systems described herein may be used to design, operate or alter larger scale bioreactors, particularly with regard to shear stress generation. In certain embodiments, data obtained from or known regarding the shear exposure patterns of cells in larger scale reactors can be simulated in a microreactor system provided by the invention in order to test and/or optimize the effects of other changes in operation and/or design of the larger scale reactor systems under more realistic shear exposure conditions. Further description of optimization and/or design of larger scale reactor systems can be found in the U.S. patent application entitled, “Methods of Providing Biochemical Analyses,” filed on even date herewith and the U.S. patent application entitled, “Microreactor Simulation of Macroreactor,”, also filed on even date herewith, both of which are hereby incorporated by reference in their entireties. Additionally, the ability to provide selected hydrodynamic shear exposure to cells and/or to control hydrodynamic shear exposure of cells is becoming increasingly important in the techniques involving tissue engineering and extracorpeal organ-assist devices.

In typical conventional small scale cell culture systems, such as for example well plates and multiple shake flasks, creating similar levels of shear stress at particular locations in multiple vessels is difficult to control. For example, placing a well plate on a conventional mixing/shaking device puts wells at a different positions and/or orientations relative to the shaker mechanism. Liquid in one well of the well plate may tend to move in a much different manner than another, thus making it difficult to generate similar shear forces within multiple wells.

In certain embodiments of the present invention, multiple sample containers are positioned and oriented similarly on the same rotation apparatus. With such a configuration, the effects of varying certain parameters at a controlled shear stress level may be tested in parallel.

In addition, certain cell culture systems capable of parallel processing and/or high-throughput such as, for example, systems including multiple well plates or shake flasks, operate in a manner such that changing parameters which affect the shear stress (for example by changing the rate of movement or shaking) can substantially change the amount of surface area at the interface between the liquid sample and gas, thereby affecting the gas exchange rate.

In certain embodiments of the present invention, shear stress may be controlled substantially independently of the gas exchange rate into or out of the liquid sample, such that creating changes in the level and/or pattern of shear stress within the liquid sample does not significantly affect the amount of surface area at the interface between the liquid sample and gas, and, therefore, does not typically substantially affect the rate of gas exchange between the liquid sample and the exterior of a reactor that contains the liquid sample.

Such embodiments may include a shear-generating element within a reactor and a control system, such as a computer-implemented process control system, in operative association with the reactor and configured for moving and/or controlling the movement of the shear-generating element via, for example, the application of external force(s) such as gravitational, centrifugal, mechanical, pneumatic, hydraulic, magnetic, and/or electrical forces.

In typical conventional systems that can allow for some control of shear forces, such as perfusion systems and rotating drum systems, relatively large volumes of liquid sample, for example in excess of 5 milliliters, may be required for operation. Additionally, in many such systems, for each liquid sample, a separate force/flow generating component is required. For example, in a perfusion system, each perfused vessel often may need a separate pump and/or controller, and, in a rotating drum system, each rotating drum assembly or small group of rotating drum assemblies may often require a separate motor and/or controller.

Certain embodiments of the present invention involve methods and systems which allow for the controllable creation of shear stress in containers, without, in many cases, the use of pumps external to the containers. In some such embodiments, an immiscible substance, such as a gas bubble, an immiscible liquid, or a solid, is used within a container as a shear-generating element such that movement of the immiscible substance creates shear stress within the container. In some embodiments, the gas bubble (or other immiscible substance) is disposed in a container, such as a reaction site container, and is moved relative to a liquid sample present within the reaction site container by reorienting the reaction site container. A density difference between the immiscible substance and the liquid sample results in the movement of the immiscible substance via gravitational and/or centrifugal forces.

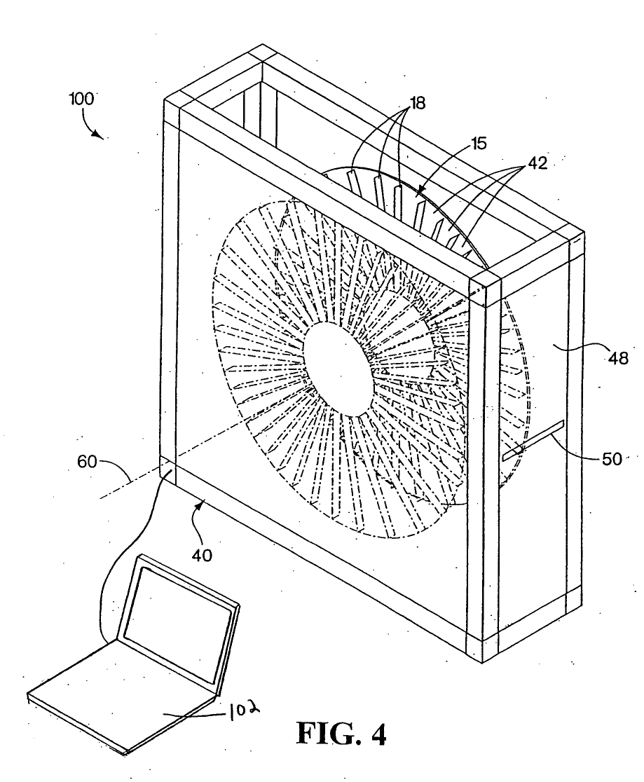

Containers used in accordance with the invention may have small volumes and/or numerous containers may be provided on a single chip such that numerous containers may be efficiently reoriented and/or controlled. In some cases, shear stress may be reproducibly created in a multiple containers, and in certain embodiments a large number of containers, using a single force-applying mechanism for creating movement of the shear-generating elements. For example, a plurality of chips, each including multiple containers including shear-generating elements, in certain embodiments is attached to a single device configured to rotate the plurality of chips (e.g., see FIG. 4). By facilitating the parallel testing of large numbers of liquid samples, the effects of shear stress on many different cells under numerous different shear exposure conditions may be accomplished efficiently.

In typical conventional cell culture reactor systems such as shake flasks and well plates, a rotating stir bar within the reactor may be used to apply shear stress to cells contained in liquid samples and/or, shear may be generated by physical agitation/motion of a container including the cell culture that includes a large enough gaseous phase in contact with the liquid to allow for liquid motion in response to the physical agitation/motion of a container sufficient to generate a desired level of mixing/agitation/shear. In such conventional systems, the ability to create strain rates and shear patterns at certain locations within the container can be difficult and/or limited.

In certain embodiments of the present invention, movement of a shear-generating element along a path of motion within a container containing a liquid sample applies shear stress to components, such as cells, within the liquid sample. Movement of a shear-generating element along a path of motion intersecting with a first location within the container and a second location within the container defines a motion that is not purely, nor, in certain embodiments, even primarily rotational, unlike rotating stir bars. As described below with reference to paths of motions in certain embodiments, the first and second locations within the container may be the same location, such that the shear-generating element moves along a path of motion that starts and ends at the same location. A path of motion may be curved and/or linear.

In certain embodiments of the invention described herein, a shear-generating element which does not comprise a surface of a container or a conduit in contact with a liquid may be employed. In some conventional perfusion systems, syringe/plunger arrangements, or other piston-type arrangements, are used to produce a liquid flow in a perfused vessel. In other conventional perfusion systems, a container having flexible and/or squeezable surfaces may be used to produce fluid flow. Certain embodiments of the present invention use freely suspended shear-generating elements and/or shear-generating elements which are attached to surfaces of a container.

In a shake flask, a well plate, or other non-enclosed reactors, the interfacial area of gas-liquid contact is a major parameter in determining gas exchange. Changes to the magnitude of shaking or other movement can substantially alter the interfacial area and thus shear stress creation and the gas exchange rate are not substantially independent.

In containers of certain embodiments of the present invention, a gas-permeable/liquid impermeable membrane is used for areas of the container surface. The permeability of this membrane can substantially control the overall exchange rate of gas between the contained liquid sample and the environment exterior to the container, typically an incubator environment. In an enclosed container that includes a gas-permeable membrane and is filled with liquid, such as those found in certain embodiments of the present invention, changes to the level of shear generation, for example by changing the rate of movement of a shear-generating element, may not, typically, substantially change the rate of gas exchange between the sample and the exterior of the container. In such embodiments, shear stress creation is substantially independent of the gas exchange rate.

In certain embodiments that include a gas bubble as a shear-generating element, changes to the levels of shear generated may result in, typically relatively modest, changes to the interfacial area of the liquid sample and the gas bubble that is present, however, many membranes that may be used for control of the exchange rate of gas to the exterior of the container may have a low enough gas permeability, for example with respect to oxygen and/or carbon dioxide, so that any changes to the interfacial area of the liquid sample and the gas bubble would not substantially change the overall gas exchange rate or the gas concentration within the liquid sample during operation.

As an example, a small difference in the oxygen exchange rate may occur when the shear-generating element is deformable (such as a gas bubble or an immiscible liquid) and changes to the rate of rotation alter the area of the membrane which the shear-generating element contacts, thereby slightly changing the available membrane area for oxygen exchange between the liquid sample and the environment exterior to the container. The changes to the overall oxygen exchange rate would, however, be small enough to be considered insubstantial when using typical oxygen permeable or semi-permeable membranes, even those having high oxygen permeabilities similar to 4-methyl-1-pentene (described below) and other similar membranes.

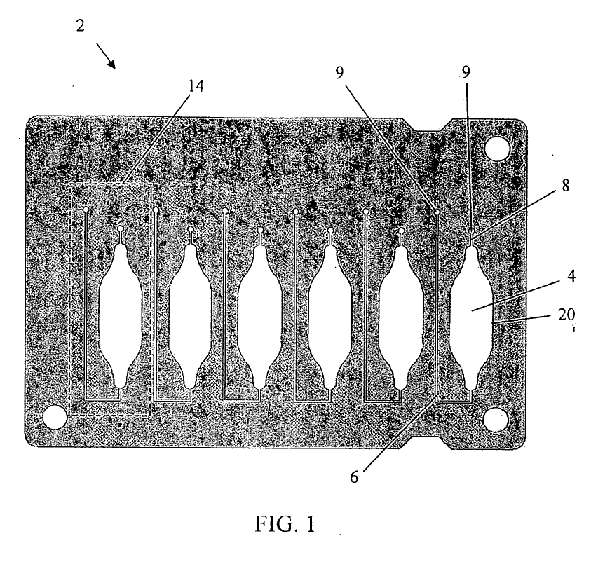

Referring now to FIG. 1, one portion of a chip which may be used in certain embodiments of the invention is illustrated schematically. The portion illustrated is a layer 2 which includes within it a series of void spaces which, when layer 2 is positioned between two adjacent layers (not shown), define a series of enclosed channels and reaction sites. An enclosed container is considered to be enclosed so long as under certain conditions of operation it is able to contain a liquid sample therein without leakage, even when the container is inverted and even if it includes inlet and outlet ports/channels, and/or includes one or more surfaces that are made from membranes which allow for the permeation of certain substances (e.g. certain gases) into or out of the container.

FIG. 1 illustrates an embodiment of a chip including six reaction sites 4 defined by reaction site containers 20. Reaction sites 4 define a series of generally aligned, elongated voids within a relatively thin, generally planar piece of material defining layer 2. Reaction sites 4 can be addressed by a series of channels including channels 8 for delivering species to reaction sites 4. In FIG. 1, each reaction site 4, along with the associated fluidic connections (e.g., channels 6 and 8, and ports 9), together define a reactor 14, as indicated by dashed lines. In FIG. 1, layer 2 contains six such reactors, each reactor having substantially the same configuration. In other embodiments, a reactor may include more than one reaction site, and/or additional channels, ports, etc. A chip can include any number of reactors, any or all of which can be identical, or any of which can be different (e.g., different sized containers, different shaped containers, different set of access channels, etc). In some embodiments, containers that do not contain reaction sites may be present as part of a reactor. For example, a reactor may include a container that does not contain a reaction site but allows for optical sensing, mixing, and/or shear generation.

Chip or reaction systems used in accordance with certain embodiments of the invention include reaction site containers that can be very small, for example, having a volume of less than about 5 milliliters, less than about 1 milliliter, or smaller—in some embodiments as small as 0.01 milliliters. In some embodiments, the reaction site includes compartments or containers that include a surface that is formed with a membrane.

In some embodiments of the invention, a reactor, a container, and/or a reaction site within a chip may be constructed and arranged to maintain an environment that promotes the growth of one or more types of living cells, for example, simultaneously. In some cases, the reaction site may be provided with fluid flow, oxygen, nutrient distribution, etc., conditions that are similar to those found in living tissue, for example, tissue from which the cells originate. Thus, the chip may be able to provide conditions that are closer to in vivo conditions than those provided by batch culture systems. In embodiments where one or more cells are used in the reaction site, the cells may be of essentially any cell type, for instance a prokaryotic cell (e.g., a bacterial cell) or a eukaryotic cell (e.g., a mammalian cell). The precise environmental conditions necessary in the reaction site for a specific cell type or types are known or may be determined by those of ordinary skill in the art using routine experimentation.

As discussed above, in certain embodiments an immiscible substance may be provided in reaction site container 20 to act as a shear-generating element. By moving an immiscible substance within reaction site container 20, the liquid sample, cells suspended in the liquid, and/or cells attached to walls of the reaction site container may be subjected to liquid motion and resulting shear exposure.

In one set of embodiments, the invention provides techniques and systems for generating and controlling the level and distribution of shear stress within a liquid sample within a container, such as reaction site containers 20 of the chips described previously. To determine the shear level and distribution created by moving a shear-generating element within a fluid sample, according to certain embodiments of the invention, computational fluid dynamics may be used. Hydrodynamic shear, τ, is a function of the fluid's viscosity, μ, and the gradient in the velocity field within the fluid, or strain rate \dot{γ}(e.g., dVx/dy):
τ=μ\dot{γ} (1)

The strain rate has units of inverse time (typically 1/s), and for reference, the viscosity of water at room temperature is around 0.001 kg/m-s.

In some embodiments, an immiscible substance may have a density that is sufficiently different from the average density of the liquid sample or carrier liquid such that changing the orientation of the container moves the immiscible substance relative to the container. This density difference may be, for example, at least 1% different than the average density of the liquid sample or carrier liquid, at least 2% different, at least 5%, at least 7%, or at least 10% different. The change in orientation causes the immiscible substance of different density to rise or sink within reaction site container 20 depending on whether the immiscible substance has a higher or lower density than the liquid sample.

As used herein, “immiscible” defines a relationship between two substances that are largely immiscible with respect to each other, but can be partially miscible. “Immiscible” substances, even if somewhat miscible with each other, will largely remain separate from each other in an observable division. For example, air and water meet this definition, in that a container of the invention containing primarily water or an aqueous solution and some air will largely phase-separate into an aqueous portion and a gas bubble or gas region, even though air is slightly soluble in water and water vapor may be present in the air. Other examples of immiscible substances, albeit those that may be somewhat miscible with each other, include oil and water, a polymeric bead and water, and the like.

The introduction of an immiscible substance within a liquid sample in container 20 may include the addition or creation of a gas bubble. The gas bubble may be introduced by partially filling the container with a liquid sample and leaving a portion as the originally present gas (typically air). In other embodiments, evaporation, cellular respiration, or the introduction of gas after filling the container may form a gas bubble.

In some cases, a container includes a predetermined gas region in fluid communication with the container. In certain embodiments, the predetermined gas region is positioned in the container. The predetermined gas region may be constructed and arranged to contain a shear-generating element when the shear-generating element is not being used to generate shear.

In some embodiments, solid elements, such as polymeric or glass beads may be included in container 20 to act as shear-generating elements. It is also possible to use a liquid that is immiscible with the liquid sample as a shear-generating element. Of course any combination of the above immiscible substances also may be used within a container. For purposes herein, the liquid sample itself or any portion thereof is not considered to be a shear-generating element.

One method, according to certain embodiments of the invention, of moving a shear-generating element involves using a rotating apparatus to change the orientation of a reactor such that a shear-generating element moves within the reactor. For example, a shear-generating element such as a gas bubble may be contained within a liquid sample container and inverting the container may cause the bubble to move from one end to the other due to buoyancy forces.

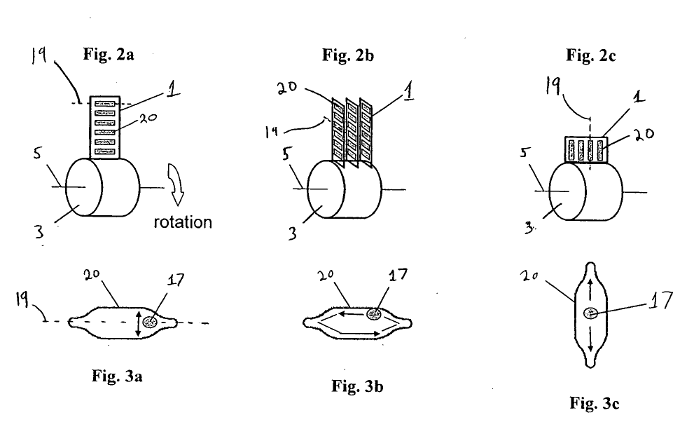

The rotating apparatuses described herein may be configured to secure the chip, article, or other substrate in any of a variety of suitable orientations. Depending on the configuration of the chip, article, or other substrate, certain such orientations may be particularly advantageous for imparting a desired level and/or pattern of shear generation. As explained in more detail below in the context of FIGS. 2a-2c, the secured orientation of the chip relative to the rotating apparatus can be relevant to the manipulation of articles comprising one or a plurality of elongate containers for the purposes of generating and/or controlling shear stress.

For example, in FIG. 2a, a chip 1, comprising a plurality of elongate containers 20, such as cell culture containers (for example, defining a predetermined reaction site), each characterized by a longitudinal direction 19, is secured to a rotating apparatus 3 configured to revolve the article about a substantially horizontal axis 5. Chip 1 is secured to apparatus 3 such that the longitudinal directions 19 of containers 20 are arranged with respect to horizontal axis 5 such that longitudinal directions 19 are substantially parallel to horizontal axis 5. As chip 1 revolves around axis 5 via the rotation of rotating apparatus 3, an immiscible substance 17 moves up and down relative to the direction of gravity which results in lateral movement (perpendicular to a longitudinal direction 19) within reaction site container 20, as shown in FIG. 3a. Immiscible substance 17 may reach the side walls of reaction site container 20 depending on the rotation rate, the relative densities and/or viscosities of immiscible substance 17 and the liquid sample, and other factors. At high rotation rates, immiscible substance 17 may not have time to move entirely to one side wall before reaction site container 20 is reversed relative to buoyancy or gravitational forces, and immiscible substance 17 moves in the opposite direction. At slower rotation rates or higher density differences, immiscible substance 17 moves faster and may reach one side wall before the reaction site container orientation is reversed.

In a the arrangement shown in FIG. 2b, chips 1 are secured to apparatus 3 such that the longitudinal directions 19 of containers 20 are arranged with respect to substantially horizontal axis 5 so that longitudinal directions 19 are substantially perpendicular to and non-intersecting with substantially horizontal axis 5. In this embodiment, immiscible substance 17 tends to follow a circuitous path within container 20 when chip 1 is revolved around axis 5, as shown in FIG. 3b. Such a path may help re-suspend cells or other species that have attached or settled along the inside perimeter of container 20. Similar to the embodiment of FIG. 2a, the extent of travel of immiscible substance 17 depends on the rotation rate and the relative densities and viscosities of immiscible substance 17 and the liquid sample.

In the configuration illustrated in FIG. 2c, chip 1 is secured to apparatus 3 such that the longitudinal directions 19 of containers 20 are arranged with respect to substantially horizontal axis 5 such that longitudinal directions 19 are substantially perpendicular to and intersect with substantially horizontal axis 5. In this configuration, immiscible substance 17 moves in an end-to-end direction 19 during rotation. Similar to the embodiments of FIGS. 2a and 2b, the extent of travel of immiscible substance 17 depends on the rotation rate, the relative densities of immiscible substance 17 and the liquid sample, and other factors.

Rotating apparatus 3 may be rotated at any suitable rate. In some embodiments, rotation rates of 2 rpm, 4 rpm, 8 rpm; 16 rpm, 32 rpm, or 65 rpm may be used, for example. In other embodiments, much higher or much lower rotation rates would be suitable depending on the species present in the liquid sample, the type and density of shear-generating element present, the level of shear stress desired, the size of the container and the rotation apparatus, and other factors. In certain embodiments, discontinuous, e.g., pulsed, rotation rates may be used. For example, apparatus 3 may be rotated at a slower rate for a length of time and then briefly rotated at a faster rate. The faster rotation rate may help to dislodge components from interior surfaces of the container and/or facilitate a more even distribution of components throughout the liquid sample. In other embodiments, the rotation of apparatus 3 may stop altogether for periods of time, for example to perform measurements of the liquid samples or components therein.

To move multiple chips or reaction site containers that contain liquid samples so as to create shear stress, a single apparatus may be used in certain embodiments of the invention. FIG. 4 shows an apparatus 100 for manipulating a chemical, biological, or biochemical sample in accordance with a variety of embodiments of the present invention. Apparatus 100, and other arrangements shown in the figures, are intended to be exemplary only. Other arrangements are possible and are embraced by the present invention. Apparatus 100 includes a housing 40 of generally rectangular solid shape. In the embodiment illustrated, housing 40 of apparatus 100 includes two, generally square, opposed major surfaces joined by four edges of rectangular shape. Housing 40 may be configured as, for example, an incubator. In some cases, housing 40 may be sufficiently enclosed so as to keep a device 15 clean, free of dust particles, within a laminar flow field, sterile, etc., depending on the application.

In certain embodiments, a control system 102 is used to operate apparatus 100 or other device(s) involved in the creation of shear stress. Control system 102 may be configured to control one or more operating parameters associated with apparatus 100, the shear-generating element, the reaction container, the chip, and/or any other components associated with an overall shear-generation system. For example, control system 102 may control the rotation rate (steady or varying) of a component of apparatus 100. Control system 102 may be attached to devices other than a rotating apparatus, for example, control system 102 may be attached to systems that can add or remove gas from the reactor container to alter the size of a gas bubble that is acting as the shear-generating element. In certain embodiments, control system 102 may have the ability to alter the orientation of the chip to the rotating apparatus.

Control system 102 may be programmed to receive feedback of various data during control operations to allow for adjustment and/or optimization of various operating parameters during operation. In certain embodiments, control system 102 may be configured to operate in conjunction with simulation software, e.g. a computational fluid dynamics software product such as FLUENT® (FLUENT USA, Lebanon N.H.), to use feedback data to develop parameter values for future operations and/or control present operating parameters.

Control system 102 may comprise a computer-implemented system. The computer implemented control system may include several known components and circuitry, including a processing unit (i.e., processor), a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as known to those of ordinary skill in the art. Further, the computer system may be a multi-processor computer system or may include multiple computers connected over a computer network.

Mounted within housing 40, on an axis 60 passing through the two, opposed major surfaces of the housing, is a device 15 for securing a plurality of individual substrates such as chips (not shown in FIG. 4) which may be constructed to contain a sample. Device 15 takes the form of a rotatable wheel with a plurality of radially outwardly extending members 18 which define, therebetween, a plurality of slots 42 within which one or more chips can be positioned. Once the chips are secured within slots 42, device 15 can be rotated, manually or automatically, about axis 60, thereby periodically inverting the chips secured in slots 42. Of course, in some embodiments, axis 60 may pass through only one of the major surfaces of the housing.

Within one face 48 of housing 40, which defines one of the edges of the housing joining the opposed major surfaces, is access port 50 through which a chip (or other substrate) can be introduced into and removed from the interior of housing 40. Access port 50 may be positioned anywhere within housing 40 that allows suitable access of chips or other substrates to apparatus 100, for example, in a side of housing 40 or on one or more major surfaces of housing 40. For the insertion of a chip into device 15 to be secured within a slot 42 of device 15, device 15 is rotated so that a desired slot is aligned with access port 50, and a chip is then inserted through access port 50 to be secured by a slot 42 within a selection region. Device 15 can be rotated to any predetermined radial orientation for aligning a desired slot 42 with access port 50, so that one or more chips can be positioned within predetermined slots 42, and their location known so the chips can be removed from device 15 such that a particular slot securing a particular chip is aligned with access port 50 for removal from device 100. The chips (or other substrates) can be inserted into and removed from housing 40 via slot 50 by essentially any suitable technique including manual operation by hand, operation by an actuator, or robotic actuation, etc. Access port 50 may be an opening in wall 48 of the housing, optionally including a flap, door, or other member that allows access port 50 to be closed when not being used to introduce or remove a chip from the housing.

In certain embodiments, instead of, or in addition to, moving container 20 so as to move a shear-generating element, e.g. by rotational inversion as discussed above, a magnetic, electrical, mechanical, pneumatic, hydraulic, and/or other force may be used. For example, a bead or beads that respond to magnetic and/or electric fields may be placed in container 20. A controlled application of a magnetic and/or electrical field may be used to move such bead(s) within container 20. Shear-generating elements that are moved by forces other than gravity/buoyancy can be the same density as the liquid within which they are contained. In some embodiments, a single controlled magnetic or electrical field may be used to move beads within numerous containers 20. Such embodiments may reduce the number of moving components of the overall system. Specifically, the ability to reduce or eliminate the movement of containers 20 while generating shear may allow for easier application of measurement techniques, such as optical measurement techniques, to the liquid samples.

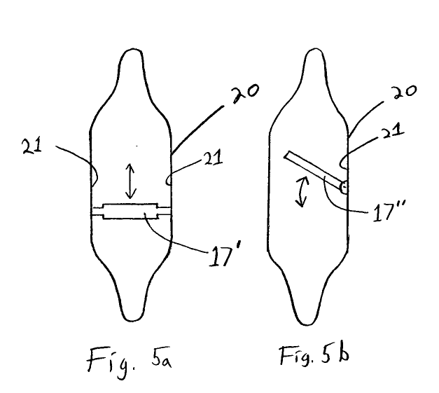

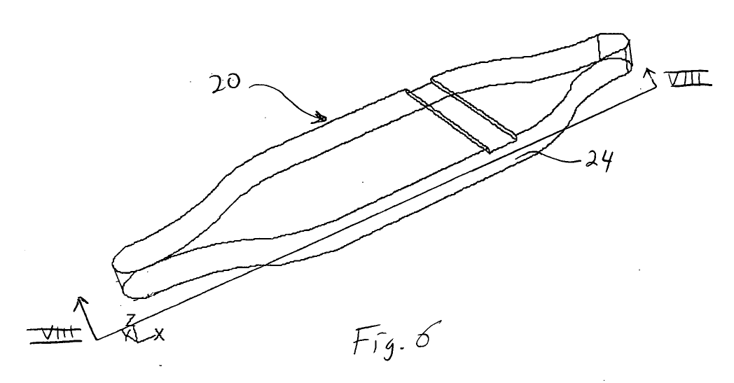

While freely suspended shear-generating elements such as gas bubbles or beads described above may be used within a container, in some embodiments, shear-generating elements which are movably attached, either directly or indirectly, to a surface of the container may be employed. For example, as shown in FIG. 5a, a movable member 17′ may be slidingly attached to a surface 21 of container 20 at two points and movable along the length of the container in response to an applied force. Movable member 17′ may be moved using any of the methods described herein, e.g., changing the orientation of the container, applying a magnetic force, and/or applying an electrical, mechanical, pneumatic, hydraulic, etc. force. For purposes herein, movable member 17′, even though movably attached to surface 21 of container 20, is not considered to be a surface of container 20 itself.

FIG. 5b illustrates an embodiment of a shear-generating element that pivots within container 20 to create shear stress. A member 17″ is attached at one location on surface 21 such that it can pivot within container 20. Member 17″ may have a density that allows for movement within a liquid sample when container 20 is moved or reoriented relative to the direction of gravity. In certain embodiments, member 17″ may react to magnetic or electrical fields, or may include components that react to such fields, so that changes to the fields and/or the orientation of container 20 with respect to those fields causes movement of member 17″.

Characteristics of the container, such as its size and/or geometry, can be varied to affect the generation and/or distribution of shear according to some embodiments of the invention. For example, as shown in FIG. 6, which is a perspective view of one embodiment of a container, the thickness of container 20 may vary along its length such that the travel path of a gas bubble or other shear-generating element traverses or is contained within a thinner portion 24 of the container. In other embodiments (not shown), the thickness of container 20 may vary continuously or discontinuously along its length and/or width. At a thinner portion 24, a gas bubble comprising the shear-generating element may deform and/or move more slowly than it otherwise would, thereby creating a different level and pattern of shear while traveling through thinner portion 24 than other portions of the container. In other embodiments (not shown) thinner portion 24 may extend along a larger percentage of the length of container 20. Conversely, in contrast to the configuration illustrated, portion 24 of container 20 could be made to be thicker than the surrounding regions of the container.

In some embodiments, one or more ports of a chip (i.e., inlet and outlet ports) are defined by “self-sealing” ports. A self-sealing port may be addressable by a needle when at least one side of the port is covered by a layer of material which, when a needle is inserted through the material and withdrawn, forms a seal generally impermeable to species such as fluids introduced into the chip via the port. In certain instances, a layer of a chip may be formed of a material that is self-sealing, i.e., the material may be penetrated by a solid object but generally regains its shape after such penetration. For example, an upper layer of a chip may be formed of an elastomeric material which may be penetrated by a mechanical device such as a needle, but which sealingly closes once the needle or other mechanical device is withdrawn.

### EXAMPLE

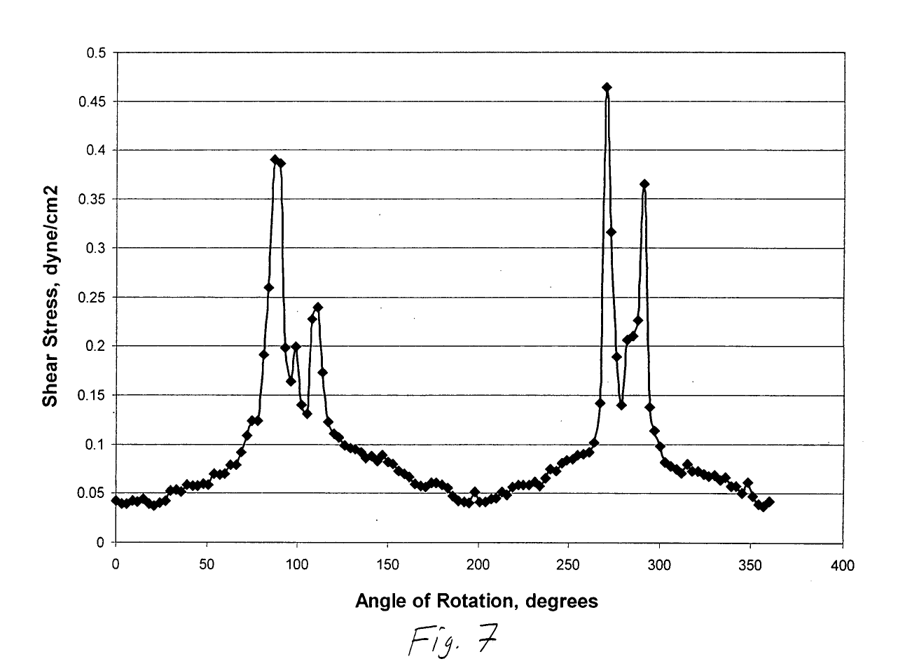

FIG. 7 shows shear stress results of a computational fluid dynamics simulation of a shear-generating element comprising a bubble traveling around a container as the container of an inventive system is rotated. The plotted shear stress was averaged over the entire volume of the container and is shown for various angles of rotation. For reference, at its starting vertical position, the container is considered to be oriented at zero degrees.

For this particular simulation, a FLUENT 6.1 computational fluid dynamics software package from FLUENT, Inc. of Lebanon, N.H. was used to run a three-dimensional strain rate simulation. The container was modeled as having a shape as in FIG. 6 and being mounted on a rotation apparatus in an orientation similar to the orientation shown in FIG. 2b and located approximately 11.9 centimeters from the axis of rotation of a rotating apparatus. The volume of the container is approximately 555 microliters, and the container has a length of 3.75 centimeters and a depth of 1.9 millimeters. The width of the container along the middle portion is 11 millimeters. Thinner portion 24 has a depth of approximately 1.54 millimeters. The simulated bubble travels along a path roughly similar to the path shown in FIG. 3b. The modeled rotation rate was 4 rpm and the bubble occupied 20% of the container volume.

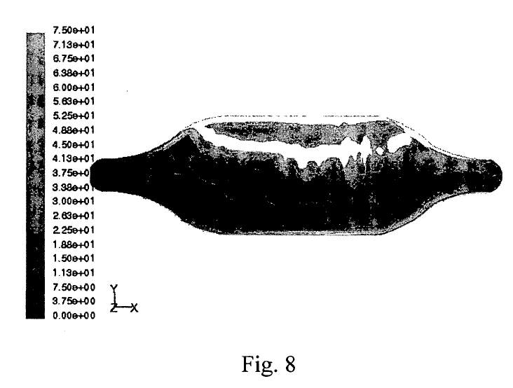

FIG. 8 shows a strain rate contour plot taken along a plane cut through line VIII-VIII of FIG. 6 at a rotation of 90 degrees based on the results of the simulation described above. The strain rates are in units of sec⁻¹. The areas of higher strain rate (white areas) within the container were located along the liquid/gas interface.

Definitions

A “chemical, biological, or biochemical reactor chip,” (also referred to, equivalently, simply as a “chip”) as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.).

A chip can be connected to or inserted into a larger framework defining an overall reaction system, for example, a high-throughput system. The system can be defined primarily by other chips, chassis, cartridges, cassettes, and/or by a larger machine or set of conduits or channels, sources of reactants, cell types, and/or nutrients, inlets, outlets, sensors, actuators, and/or controllers. Typically, the chip can be a generally flat or planar article (i.e., having one dimension that is relatively small compared to the other dimensions); however, in some cases, the chip can be a non-planar article, for example, the chip may have a cubical shape, a curved surface, a solid or block shape, etc.

As used herein, a “reaction site” is defined as a site within a reactor that is constructed and arranged to produce a physical, chemical, biochemical, and/or biological reaction during use of the chip or reactor. More than one reaction site may be present within a reactor or a chip in some cases. The reaction may be, for example, a mixing or a separation process, a reaction between two or more chemicals, a light-activated or a light-inhibited reaction, a biological process, and the like. In certain embodiments, the reaction site may also include one or more cells and/or tissues.

The volume of the reaction site can be very small in certain embodiments and may have any convenient size. Specifically, the reaction site may have a volume of less than one liter, less than about 100 ml, less than about 10 ml, less than about 5 ml, less than about 3 ml, less than about 2 ml, less than about 1 ml, less than about 500 microliters, less than about 300 microliters, less than about 200 microliters, less than about 100 microliters, less than about 56 microliters, less than about 30 microliters, less than about 20 microliters or less than about 10 microliters in various embodiments. The reaction site may also have a volume of less than about 5 microliters, or less than about 1 microliter in certain cases. In another set of embodiments, the reaction site may have a dimension that is 2 millimeters deep or less, 500 microns deep or less, 200 microns deep or less, or 100 microns deep or less.

“Elongate(d),” as used herein when referring to a chamber or substrate or container or predetermined reaction site of an article, refers to such chamber or substrate or container or predetermined reaction site having a perimetric shape, e.g., of an outer boundary or container, that is characterized by there being a first straight line segment, contained within the outer boundary/container, connecting two points on the outer boundary/container and passing through the geometric center of the chamber or substrate or container or predetermined reaction site, that is substantially longer than a second straight line segment, perpendicular to the first line segment, contained within the outer boundary/container, connecting two points on the outer boundary/container—other than the same two points connected by the first line segment—and passing through the geometric center of the chamber or substrate or container or predetermined reaction site. For example, if the article is a planar chip comprising a volumetric container defining a predetermined reaction site characterized by a thickness, measured in a direction perpendicular the plane of the chip and a length and width, measured in mutually perpendicular directions both parallel to the plane of the chip, the predetermined reaction site would be “elongate,” if the length substantially exceeded the width (e.g., as would be the case for a thin, rectangular or ellipsoidal, tear-shaped, etc., predetermined reaction site). A direction co-linear with the longest such straight line segment, contained within the outer boundary/container, connecting two points on the outer boundary/container and passing through the geometric center of the chamber or substrate or container or predetermined reaction site for an elongate chamber, substrate, container or predetermined reaction site is referred to herein as the “longitudinal direction” of the chamber or substrate or container or predetermined reaction site.

As used herein, a “membrane” is a thin sheet of material, typically having a shape such that one of the dimensions is substantially smaller than the other dimensions, that is permeable to at least one substance in an environment to which it is or can be exposed. In some cases, the membrane may be generally flexible or non-rigid. As an example, a membrane may be a rectangular or circular material with a length and width on the order of millimeters, centimeters, or more, and a thickness of less than a millimeter, and in some cases, less than 100 microns, less than 10 microns, or less than 1 micron or less. The membrane may define a portion of a reaction site and/or a reactor, or the membrane may be used to divide a reaction site into two or more portions, which may have volumes or dimensions which are substantially the same or different. For example, a reaction site may be divided into three portions, four portions, or five portions. For instance, a reaction site may be divided into a first cell culture portion and a second cell culture portion flanking a first reservoir portion and two additional reservoir portions, one of which is separated by a membrane from the first cell culture portion and the other of which is separated by a membrane from the second cell culture portion. One or more membranes may also define one or more walls of a reaction site container. For instance, in one embodiment, a first membrane (e.g., a gas permeable vapor impermeable membrane) defines a first wall of a reaction site container. In another embodiment, a second membrane (e.g., a gas permeable vapor impermeable membranes) defines a second wall of the reaction site container. Non-limiting examples of substances to which the membrane may be permeable to include water, O₂, CO₂, or the like. As an example, a membrane may have a permeability to water of less than about 1000 (g micrometer/m²·day), 900 (g micrometer/m²·day), 800 (g micrometer/m²·day), 600 (g micrometer/m² day) or less; the actual permeability of water through the membrane may also be a function of the relative humidity in some cases. As another example, a membrane may have a permeability to oxygen of about 0.061 mol O₂/(day·m²·atm) or greater.

Some membranes may be semipermeable membranes, which those of ordinary skill in the art will recognize to be membranes permeable with respect to at least one species, but not readily permeable with respect to at least one other species. For example, a semipermeable membrane may allow oxygen to permeate across it, but not allow water vapor to do so, or may allow water vapor to permeate across it, but at a rate that is at least an order of magnitude less than that for oxygen. Or a semipermeable membrane may be selected to allow water to permeate across it, but not certain ions. For example, the membrane may be permeable to cations and substantially impermeable to anions, or permeable to anions and substantially impermeable to cations (e.g., cation exchange membranes and anion exchange membranes). As another example, the membrane may be substantially impermeable to molecules having a molecular weight greater than about 1 kilodalton, 10 kilodaltons, or 100 kilodaltons or more. In one embodiment, the membrane may be impermeable to cells, but be chosen to be permeable to varied selected substances; for example, the membrane may be permeable to nutrients, proteins and other molecules produced by the cells, waste products, or the like. In other cases, the membrane may be gas impermeable. Some membranes may be transparent to particular light (e.g. infrared, UV, or visible light; light of a wavelength with which a device utilizing the membrane interacts; visible light if not otherwise indicted). Where a membrane is substantially transparent, it absorbs no more than 50% of light, or in other embodiments no more than 25% or 10% of light, as described more fully herein. In some cases, a membrane may be both semipermeable and substantially transparent.

In some cases, the material of the membrane may include monomers or polymers, or a co-polymer, a polymer blend, a multi-layered structure comprising polymers in at least one layer, etc. Non-limiting examples of polymers that may be used within the membrane material include polyfluoroorganic materials such as polytetrafluoroethylenes (e.g., such as those marketed under the name TEFLON® by DuPont of Wilmington, Del., for example, TEFLON® AF) or certain amorphous fluoropolymers; polystyrenes; polypropylenes (“PP”); silicones such as polydimethylsiloxanes; polysulfones; polycarbonates; acrylics such as polymethyl acrylate and polymethyl methacrylate; polyethylenes such as high-density polyethylenes (“HDPE”), low-density polyethylenes (“LDPE”), linear low-density polyethylenes (“LLDPE”), ultra low-density polyethylenes (“ULDPE”) etc.; PET; polyvinylchloride (“PVC”) materials; nylons; a thermoplastic elastomer; poly(1-trimethlsilyl-1-propyne) (“PTMSP”); and the like. Another example is poly(4-methylpentene-1) or poly(4-methyl-1-pentene) or poly(4-methyl-2-pentyne) (“PMP”). Examples of PMPs include those marketed under the name TP™ by Mitsui Plastics (White Plains, N.Y.). As still another example, membrane material may include poly(4-methylhexene-1), poly(4-methylheptene-1), poly(4-methyloctene-1), etc. In some cases, these materials may be copolymerized and/or in a polymer blend in association with the polymers as described above.

In some embodiments, two or more components of the chip may be joined using an adhesive material. As used herein, an “adhesive material” is given its ordinary meaning as used in the art, i.e., an auxiliary material able to fasten or join two other materials together. For instance, an adhesive may be used to bind a membrane to a substrate layer defining a reaction site. Non-limiting examples of adhesive materials suitable for use with the invention include silicone adhesives such as pressure-sensitive silicone adhesives, neoprene-based adhesives, and latex-based adhesives. The adhesive may be applied to one or more components of the chip using any suitable method, for example, by applying the adhesive to a component of the chip as a liquid or as a semi-solid material such as a viscoelastic solid. For example, in certain embodiments, the adhesive may be applied to the component(s) using transfer tape (e.g., a tape having adhesive material attached thereto, such that, when the tape is applied to the component, the adhesive, or at least a portion of the adhesive, remains attached to the component when the tape is removed from the component). In one set of embodiments, the adhesive may be a pressure-sensitive adhesive, i.e., the material is not normally or substantially adhesive, but becomes adhesive and/or increases its adhesive strength under the influence of pressure, for example, a pressure greater than about 6 atm or about 13 atm (about 100 psi or about 200 psi). Non-limiting examples of pressure-sensitive adhesives include AR Clad 7876 (available from Adhesives Research, Inc., Glen Rock, Pa.) and Trans-Sil Silicone PSA NT-1001 (available from Dielectric Polymers, Holyoke, Mass.).

In some embodiments, the chip may be constructed and arranged such that one or more reaction sites can be defined, at least in part, by two or more components fastened together as previously described (i.e., with or without an adhesive). In some cases, a reaction site may be free of any adhesive material adjacent to or otherwise in contact with one or more surfaces defining the reaction site, and this can be advantageous, for instance, when an adhesive might otherwise leach into fluid at the reaction site. Of course, an adhesive may be used elsewhere in the chip, for example, in other reaction sites. Similarly, in certain cases, a reaction site may be constructed using adhesive materials, such that at least a portion of the adhesive material used to construct the reaction site remains within the chip such that it is adjacent to or otherwise remains in contact with one or more surfaces defining the reaction site. Of course, other components of the chip may be constructed without the use of adhesive materials, as previously discussed.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An apparatus for performing a biological or biochemical reaction having the ability to apply shear stress to a component of a liquid sample, comprising:
a biological or biochemical reactor comprising a container having a volume of less than about 2 mL, the container containing a liquid sample; and
a shear-generating element, which does not comprise a surface of a container or a conduit in contact with a liquid, the shear-generating element being contained within the apparatus and constructed and arranged so that the entire shear-generating element moves along a selected path of motion intersecting a first location within the apparatus and a second location within the apparatus, with or without rotational movement.

2. An apparatus as in claim 1, wherein the shear-generating element is contained and movable within the container containing the liquid sample.

3. An apparatus as in claim 1, wherein the container is constructed and arranged to facilitate cell cultivation.

4. An apparatus as in claim 3, wherein the container is constructed and arranged to facilitate mammalian cell cultivation.

5. An apparatus as in claim 1 further comprising:
a first gas permeable, liquid vapor impermeable membrane defining a first wall of the container.

6. An apparatus as in claim 1, wherein the shear-generating element has an average density at least 1% different from the average density of the liquid sample.

7. An apparatus as in claim 1, wherein the shear-generating element has an average density at least 5% different from the average density of the liquid sample.

8. An apparatus as in claim 1, wherein the container has a volume of less than about 1.3 mL.

9. An apparatus as in claim 1, wherein changes in the movement of the shear-generating element that create changes in a level or pattern of shear stress within the liquid sample do not significantly affect gas exchange between the liquid sample and the exterior of the reactor.

10. An apparatus as in claim 1, wherein the shear-generating element is a gas bubble contained within the apparatus.

11. An apparatus as in claim 1, wherein the shear-generating element is a gas bubble contained and movable within the container containing the liquid sample.

12. An apparatus as in claim 1, further comprising a control system configured to control the movement of the shear-generating element to facilitate the creation of a reproducible and controllable level of shear stress at a selected location within the liquid sample.

13. An apparatus as in claim 1, wherein the gas permeable, liquid vapor impermeable membrane has an oxygen permeability of greater than or equal to 0.061 mol O₂/(day·m2·atm).

14. An apparatus as in claim 13, wherein the membrane has an oxygen permeability of less than or equal to 0.6 mol O₂/(day·m2·atm).

15. An apparatus as in claim 1, wherein a second gas permeable, liquid vapor impermeable membrane defines a second wall of the container.

16. An apparatus as in claim 2, wherein the thickness of the container varies along the selected path of motion of the shear-generating element.

17. An apparatus as in claim 1, further comprising a plurality of containers each having a volume of less than about 2 mL and each containing a liquid sample.

18. An apparatus as in claim 17, wherein the plurality of containers are present on a chip, and the chip is constructed and arranged to enable it to be stably connected in a selected orientation with respect to other, similar chips in the apparatus.

19. An apparatus as in claim 1, wherein the shear-generating element is a solid element.

20. An apparatus as in claim 1, wherein the shear-generating element is a liquid immiscible in the liquid sample.

21. An apparatus as in claim 1, further comprising a rotating apparatus to which the container is attached.

22. An apparatus as in claim 1, wherein the chip further comprises a predetermined gas region in fluid communication with the container, in which the shear-generating element can be positioned when the shear-generating element is not being used to generate shear.

23. An apparatus as in claim 1, further comprising an inlet port, an outlet port, and a self-sealing elastomeric material defining portions of the inlet port and the outlet port.

24. An apparatus as in claim 1, wherein the container is defined by a void in a substrate layer.

25. An apparatus as in claim 5, wherein an adhesive layer binds the gas permeable vapor impermeable membrane to the substrate layer.

26. An apparatus for performing a biological or biochemical reaction having the ability to apply shear stress to a component of a liquid sample, comprising:
a biological or biochemical reactor comprising a container containing a liquid sample; and
a shear-generating element, which does not comprise a surface of a container or a conduit in contact with a liquid, the shear-generating element being contained within the container and constructed and arranged so that the entire shear-generating element moves along a selected path of motion intersecting a first location within the container and a second location within the container, wherein
changes in the movement of the shear-generating element within the container that create changes in a level or pattern of shear stress in the liquid sample do not significantly affect gas exchange between the liquid sample and the exterior of the reactor.

27. An apparatus as in claim 26, further comprising a first gas permeable, liquid vapor impermeable membrane defining a first wall of the container.

28. An apparatus as in claim 27, further comprising an inlet port, an outlet port, and at least one microfluidic channel in fluid communication with the container.

29. An apparatus as in claim 27, wherein the container is constructed and arranged to maintain at least one living mammalian cell.

30. An apparatus as in claim 26, wherein the shear-generating element has an average density at least 1% different from the average density of the liquid sample.

31. An apparatus as in claim 26, wherein the container has a volume of less than about 5 mL.

32. An apparatus as in claim 26, wherein the shear-generating element is a gas bubble.

33. An apparatus as in claim 26, further comprising a control system configured to control the movement of the shear-generating element to facilitate the creation of the reproducible and controllable level of shear stress at a selected location within the liquid sample.

34. An apparatus as in claim 26, wherein the shear-generating element is a solid element attached to the container.

35. An apparatus as in claim 34, wherein the shear-generating element is slidingly attached to the container.

36. An apparatus as in claim 26, wherein the shear-generating element is a liquid immiscible in the liquid sample.

37. An apparatus for performing a biological or biochemical reaction having the ability to apply shear stress to a component of a liquid sample, comprising:
a biological or biochemical reactor comprising a container, the container containing a liquid sample;
a shear-generating element within the apparatus that is movable within the apparatus upon inversion of the apparatus; and
a control system configured to control movement of the shear-generating element to facilitate creation of a reproducible and controllable level of shear stress at a selected location within the liquid sample.

38. An apparatus as in claim 37, wherein the container has a volume of less than 5 mL.

39. An apparatus as in claim 37, wherein the container has a volume of greater than 0.01 mL and less than 3 mL.

40. An apparatus as in claim 37, wherein the container has a volume of greater than 0.5 mL and less than 3 mL.

41. An apparatus as in claim 37, further comprising a first gas permeable, liquid vapor impermeable membrane defining a first wall of the container.

42. An apparatus as in claim 37, wherein the shear-generating element has an average density at least 1% different from the average density of the liquid sample.

43. An apparatus as in claim 37, wherein the shear-generating element is a gas bubble.

44. An apparatus as in claim 37, wherein the shear-generating element is a gas bubble contained and movable within the container containing the liquid sample.

45. A method of applying shear stress to a biological or biochemical component of a liquid sample contained within a container, comprising:
moving and/or controlling movement of a shear-generating element within a container containing a liquid sample, the movement of the shear-generating element occurring upon inversion of the container, wherein the movement applies a reproducible and controllable level of shear stress to a biological or biochemical component at a selected location within the liquid sample.

46. A method as in claim 45, wherein the container is capable of maintaining at least one living cell.

47. A method as in claim 45, wherein the container is capable of maintaining at least one living mammalian cell.

48. A method as in claim 45, a gas permeable, liquid vapor impermeable membrane defines a first wall of the container.

49. A method as in claim 45, wherein the movement applies a preselected level of shear stress at the selected location within the liquid sample.

50. A method as in claim 49, further comprising an additional and separate act of moving and/or controlling movement of the shear-generating element within the container containing a liquid sample, the movement of the shear-generating element occurring upon inversion of the container, wherein the additional and separate movement applies a different preselected level of shear stress at the selected location within the liquid sample.

51. A method as in claim 45, wherein the shear-generating element is a gas bubble.

52. A method as in claim 45, wherein the shear-generating element is a liquid immiscible in the liquid sample.

53. A method as in claim 45, wherein the container is less than approximately 5 mL.

54. A method as in claim 45, wherein the container is less than approximately 1 mL.

55. A method as in claim 45, wherein the container is less than approximately 0.01 mL.

56. A method as in claim 45, wherein moving and/or controlling movement of the shear-generating element comprises rotating the container.

57. A method as in claim 56, wherein rotating the container comprises rotating the container about an axis that is external to the container.

58. A method as in claim 56, further comprising rotating the container using discontinuous rotation rates.

59. An apparatus for performing a biological or biochemical reaction having the ability to apply shear stress to a component of a liquid sample, comprising:
a biological or biochemical reactor comprising a container configured to contain a liquid sample, a surface of the container comprising a membrane having an oxygen permeability of greater than or equal to 0.061 mol O₂/(day·m2·atm); and
a shear-generating element contained within the container and constructed and arranged so that the entire shear-generating element moves along a selected path of motion intersecting a first location within the container and a second location within the container during operation when the container contains the liquid sample.

60. An apparatus as in claim 59, wherein the container has a volume of less than 5 mL.

61. An apparatus as in claim 60, wherein the container contains the liquid sample and wherein the shear-generating element is a gas bubble.

62. An apparatus as in claim 59, wherein the shear-generating element is a solid element.

63. A method as in claim 59, wherein the container contains the liquid sample and wherein the shear-generating element is a liquid immiscible in the liquid sample.

64. An apparatus as in claim 59, wherein the membrane has an oxygen permeability of less than or equal to 69.7 mol O₂/(day·m2·atm).

65. An apparatus as in claim 59, wherein the membrane has an oxygen permeability of less than or equal to 140 mol O₂/(day·m2·atm).

66. An apparatus as in claim 59, further comprising a control system configured to control the movement of the shear-generating element relative to the container so as to create a reproducible and controllable level of shear stress at a selected location within the liquid sample.

67. A method of applying shear stress to a biological or biochemical component of a liquid sample comprising:
moving an entire shear-generating element, freely suspended within an apparatus, along a selected path of motion intersecting a first location within the apparatus and a second location within the apparatus to apply a reproducible and controllable level of shear stress to a biological or biochemical component at a selected location within a liquid sample, wherein the shear-generating element is either a gas or a liquid.

68. A method as in claim 67, wherein the liquid sample is contained within a reaction site container that is capable of maintaining at least one living cell.

69. A method as in claim 68, wherein the at least one living cell is a mammalian cell.

70. A method as in claim 67, further comprising a gas permeable, liquid vapor impermeable membrane defining a first wall of the container.

71. A method as in claim 67, wherein the moving the shear-generating element a preselected level of shear stress at the selected location within the liquid sample.

72. A method as in claim 67, wherein the shear-generating element is a gas bubble.

73. A method as in claim 67, wherein the shear-generating element is a liquid immiscible in the liquid sample.

74. A method as in claim 67, wherein moving the shear-generating element comprises changing the orientation of the apparatus relative to the direction of gravity.

75. A method as in claim 67, wherein moving the shear-generating element comprises applying a magnetic field to the shear-generating element.

76. A method as in claim 67, wherein moving the shear-generating element comprises applying an electrical field to the shear-generating element.

77. A method as in claim 67, wherein the shear-generating element is freely suspended within a container having a volume of less than about 2 mL.

78. A method as in claim 67, further comprising:
receiving feedback of at least one measurement from the liquid sample; and
adjusting at least one control parameter of the apparatus in response to the measurement.

79. A method as in claim 67, further comprising:
receiving feedback of at least one measurement from the liquid sample; and
operating simulation software to determine at least one parameter value for controlling the apparatus.

80. An apparatus for performing a biological or biochemical reaction having the ability to apply shear stress to a component of a liquid sample, comprising:
a biological or biochemical reactor comprising a container having a volume of less than about 2 mL and containing a liquid sample; and
a shear-generating element, which does not comprise a surface of a container or a conduit in contact with a liquid, the shear-generating element being contained within the container and constructed and arranged for pivoting movement within the container, the pivoting movement creating a reproducible and controllable level of shear stress at a selected location within the liquid sample.

81. An apparatus as in claim 80, wherein the shear-generating element is a member having an end that is pivotally attached to an interior surface of the container.
